SEQUENCE LISTING (7 sequences)

SEQ ID NO: 1 - protein, 9 aa, Homo sapiens
SEVEEPLSQ

SEQ ID NO: 2 - protein, 10 aa, Homo sapiens
ESEVEEPLSQ

SEQ ID NO: 3 - protein, 10 aa, Homo sapiens
SEVEEPLSQE

SEQ ID NO: 4 - protein, 11 aa, Homo sapiens
QESEVEEPLSQ

SEQ ID NO: 5 - protein, 11 aa, Homo sapiens
ESEVEEPLSQE

SEQ ID NO: 6 - protein, 11 aa, Homo sapiens
SEVEEPLSQES

SEQ ID NO: 7 - protein, 186 aa, Homo sapiens
MSPKPRASGPPAKATEAGKRKSSSQPSPSDPKKKTTKVAKKGKAVRRGRRGKKGAATKMAAVTAPEAESGPAAPGPSDQPSQELPQHELPPEEPVSEGTQHDPLSQESELEEPLSQESEVEEPLSQESQVEEPLSQESEVEEPLSQESQVEEPLSQESEVEEPLSQESQVEEPLSQESEMEELPSV

(19) United States
(12) Patent Application Publication
(10) Pub. No.: US 2022/0347279 A1
(43) Pub. Date: Nov. 3, 2022

(54) VCX/Y PEPTIDES AND USE THEREOF

(71) Applicant: BOARD OF REGENTS, THE UNIVERSITY OF TEXAS SYSTEM, Austin, TX (US)

(72) Inventors: Cassian YEE, Houston, TX (US); Ke PAN, Houston, TX (US)

(73) Assignee: BOARD OF REGENTS, THE UNIVERSITY OF TEXAS SYSTEM, Austin, TX (US)

(21) Appl. No.: 17/763,906

(22) PCT Filed: Oct. 2, 2020
(86) PCT No.: PCT/US2020/053929

§ 371 Date: Mar. 25, 2022

Related U.S. Application Data
(60) Provisional application No. 62/910,128, filed on Oct. 3, 2019.

(51) Int. Cl. A61K 39/00; C07K 14/47; A61K 39/395; A61K 31/675; A61K 31/7076; A61P 35/00

(52) U.S. Cl. CPC A61K 39/0011; C07K 14/47; A61K 39/3955; A61K 31/675; A61K 31/7076; A61P 35/00; A61K 38/00

(57) ABSTRACT

Provided herein are tumor-antigen VCX/Y specific peptides and engineered VCX/Y specific T cell receptors. Also provided herein are methods of generating VCX/Y-specific immune cells and their use for the treatment of cancer. In addition, the

This application claims priority to U.S. Provisional Patent Application Ser. No. 62/910,128, filed Oct. 3, 2019, which is incorporated by reference herein in its entirety.

### BACKGROUND

### 1. Field

The present disclosure relates generally to the fields of immunology and medicine. More particularly, it concerns tumor antigen peptides and uses thereof for the treatment of cancer.

### 2. Description of Related Art

T cell-based therapies have shown significant promise as a method for treating many cancers; unfortunately, this approach has also been hindered by a paucity of immunogenic antigen targets for common cancers and potential toxicity to non-cancerous tissues. These T cell based therapies can include ACT (adoptive cell transfer) and vaccination approaches. ACT generally involves infusing a large number of autologous activated tumor-specific T cells into a patient, e.g., to treat a cancer. ACT has resulted in therapeutic clinical responses in melanoma patients (Yee 2002; Dudley 2002; Yee 2014). Generally, to develop effective anti-tumor T cell responses, the following three steps are normally required: priming and activating antigen-specific T cells, migrating activated T cells to tumor site, and recognizing and killing tumor by antigen-specific T cells. The choice of target antigen is important for induction of effective antigen-specific T cells.

While several tumor-associated antigens have been identified for melanoma and a handful of other solid tumor malignancies, there are few immunogenic targets for pancreatic, ovarian, gastric, lung, cervical, breast, and head and neck cancer. Identification and validation of novel epitopes and target antigens for these common and difficult to treat malignancies is warranted.

### SUMMARY

The present disclosure provides, in at least some embodiments, methods and compositions related to peptides from the Variable Charge X-Linked/Y-linked (VCX/Y) family (e.g., VCX1, VCX2, VCX3A, VCX3B, and VCY), including peptides that may be used in adoptive T cell therapies. In some embodiments, the peptides may be used to expand VCX/Y-specific T cells in vitro that are administered to a mammalian subject, such as a human patient, to treat a disease (e.g., a cancer). In further embodiments, the T cells are genetically engineered to express T cell receptors (TCRs) with antigenic specificity for VCX/Y. In other embodiments, the peptides may be administered to a mammalian subject to induce an immune response or vaccinate the subject against the peptide, and such an immune response may be useful to treat or reduce the chances of getting or relapsing from a disease, such as a cancer.

In one embodiment, the present disclosure provides an isolated VCX/Y (e.g., VCX3A) peptide of 35 amino acids in length or less comprising an amino acid sequence having at least 90% sequence identity to SEQ ID NO: 1 (SEVEEPLSQ). In some aspects, the peptide comprises an amino acid sequence having at least 91, 92, 93, 94, 95, 96, 97, 98, 99, or 100 percent sequence identity to SEQ ID NO:1. In particular embodiments, the peptide is capable of inducing cytotoxic T lymphocytes (CTLs)

In certain aspects, the peptide is 30 amino acids in length or less, such as 29, 28, 27, 26, 25, 24, 23, 22, 21, 20, 19, 18, 17, 16, 15, 14, 13, 12, 11, or 10 amino acids in length.

In another embodiment, there is provided a pharmaceutical composition comprising the isolated VCX/Y peptide of the embodiments and a pharmaceutical carrier. In some aspects, the pharmaceutical composition is formulated for parenteral administration, intravenous injection, intramuscular injection, inhalation, or subcutaneous injection, as examples only. In certain aspects, the peptide is comprised in a liposome, nanoparticle (e.g., lipid-containing nanoparticle), or in a lipid-based carrier. In some aspects, the pharmaceutical preparation is formulated for injection or inhalation as a nasal spray.

A further embodiment provides an isolated nucleic acid encoding the VCX/Y peptide of the embodiments. Also provided herein is a vector comprising a contiguous sequence consisting of or comprising the nucleic acid encoding the VCX/Y peptide.

In yet another embodiment, there is provided a method of promoting an immune response in a subject, comprising administering an effective amount of the VCX/Y peptide of the embodiments to the subject, wherein the peptide induces antigen-specific T cells in the subject. In some aspects, the subject is diagnosed with cancer or is at risk for cancer, including at a risk higher than the general population, for example. In certain aspects, the cancer is pancreatic, ovarian, gastric, or breast cancer. In particular aspects, the subject is a human.

In additional aspects, the method further comprises administering at least a second anti-cancer therapy. In some aspects, the second anti-cancer therapy is selected from the group consisting of a chemotherapy, a radiotherapy, an immunotherapy, or a surgery. In particular aspects, the immunotherapy is an immune checkpoint inhibitor. In one specific aspects, the immune checkpoint inhibitor is an anti-PD1 monoclonal antibody.

A further embodiment provides a method of producing VCX/Y-specific T cells comprising obtaining a starting population of T cells, and contacting the starting population of T cells with the VCX/Y peptide of the embodiments, thereby generating VCX/Y-specific T cells. In some aspects, contacting is further defined as co-culturing the starting population of T cells with antigen presenting cells (APCs), wherein the APCs present the VCX/Y peptide of the embodiments on their surface. In particular aspects, the APCs are dendritic cells. In some aspects, the starting population of T cells are CD8⁺T cells. In certain aspects, the T cells are cytotoxic T lymphocytes (CTLs). In some aspects, obtaining comprises isolating the starting population of T cells from peripheral blood mononuclear cells (PBMCs). Also provided herein is a pharmaceutical composition comprising the VCX/Y-specific T cells produced by the methods herein.

An even further embodiment provides an antigen receptor, such as a T cell receptor (TCR) or chimeric antigen receptor (CAR), with antigenic specificity for VCX/Y. Another embodiment provides T cells engineered to express a VCX/Y-specific TCR and/or VCX/Y-specific CAR.

Another embodiment provides a method of treating cancer in a subject comprising administering an effective amount of the VCX/Y-specific T cells of the embodiments to the subject. In some aspects, the cancer is thymoma, bladder cancer, uterine carcinoma, melanoma, sarcoma, cervix cancer, or head and neck cancer. In particular aspects, the subject is a human. In some aspects, the cells are autologous or allogeneic with respect to the recipient individual. In some aspects, the subject is determined to have cancer cells that express VCX/Y, although in other cases it is unknown if the subject has cancer cells that express VCX/Y.

In some aspects, the host cell is a T cell, peripheral blood lymphocyte, NK cell, invariant NK cell, NKT cell, mesenchymal stem cell (MSC), induced pluripotent stem (iPS) cell, or mixture thereof. In certain aspects, the host cell is isolated from the umbilical cord. In some aspects, the host cell is autologous or allogeneic with respect to a recipient individual. In certain aspects, the T cell is a CD8⁺T cell, CD4⁺T cell, γδ T cell, or a mixture thereof.

In certain aspects, the method further comprises lymphodepletion of the subject prior to administration of the antigen-specific T cells. In some aspects, lymphodepletion comprises administration of cyclophosphamide and/or fludarabine.

In some aspects, the method further comprises administering at least a second therapeutic agent. In certain aspects, the at least a second therapeutic agent comprises chemotherapy, immunotherapy, surgery, radiotherapy, and/or biotherapy. In particular aspects, the immunotherapy is one or more immune checkpoint inhibitors. In some specific aspects, the immune checkpoint inhibitor is an anti-PD1 monoclonal antibody.

In certain aspects, the VCX/Y-specific T cells and/or the at least a second therapeutic agent are administered intravenously, intraperitoneally, intratracheally, intratumorally, intramuscularly, endoscopically, intralesionally, percutaneously, subcutaneously, regionally, or by direct injection or perfusion.

In some aspects, the subject is determined to have cancer cells that express a protein of the VCX/Y family. In particular aspects, the protein is VCX3A.

Other objects, features and advantages of the present disclosure will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The subject matter of the disclosure may be better understood by reference to one or more of these drawings in combination with the description of illustrative embodiments presented herein.

FIG. 1: Overlap peptide library screening for the recognition target peptide of VCX54 TCR-T, as one example. Sequence in its entirety is SEQ ID NO:7.

FIG. 2: VCX118 peptide cross reaction detection.

FIG. 3: Peptide titration killing assay.

FIG. 4: VCX118 peptide HLA-A2 binding assay. In the separate bar groupings of three, from left to right the bars represent 100 ug, 30 ug, and 10 ug.

FIG. 5: VCX54 TCR-T recognition to different length of longer peptide comprising VCX118.

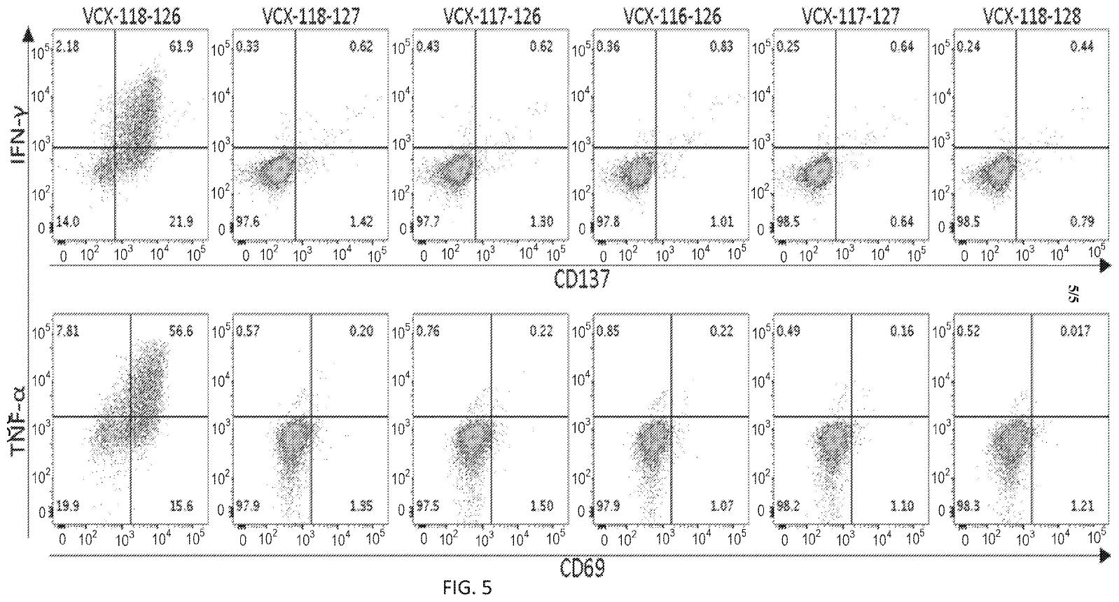

### DESCRIPTION OF ILLUSTRATIVE EMBODIMENTS

For individuals with many different cancer types, T cell-based immunotherapies represent a promising approach with proven efficacy. However, antigen-specific T cell therapy for most cancer types is not feasible because of the lack of tumor-associated antigens currently known, which has stalled their clinical development. Studies in the present disclosure identified novel VCX/Y family-derived peptide epitopes found in all of the VCX/Y family members including VCX1, VCX2, VCX3A, VCX3B, and VCY. Using the peptide epitopes, antigen-specific cytotoxic T lymphocytes (CTLs) were generated from patient peripheral blood mononuclear cells (PBMCs) that recognized the endogenously- presented antigen on allogeneic tumor cell lines, leading to tumor cell killing. Thus, these antigen-specific CTLs may be used to target solid cancers (e.g., pancreatic, ovarian, gastric, and breast cancer).

Accordingly, the present disclosure provides tumor antigen-specific peptides, such as to tumor antigen VCX/Y, for the use as immunotherapy, or related to therapy, for the treatment of a cancer. An exemplary VCX/Y peptide, VCX118 (e.g. comprising SEQ ID NO:1), is disclosed herein, the sequence of which is shared with all VCX/Y family members including VCX1, VCX2, VCX3A, VCX3B, and VCY. For example, a tumor antigen-specific peptide may be contacted with or used to stimulate a population of T cells to induce proliferation of the T cells that recognize or bind the tumor antigen-specific peptide. In other embodiments, a VCX/Y-specific peptide of the present disclosure may be administered to a subject, such as a human patient, to enhance the immune response of the subject against a cancer.

A VCX/Y-specific peptide may be included in an active immunotherapy (e.g., a cancer vaccine) or a passive immunotherapy (e.g., an adoptive immunotherapy). Active immunotherapies include immunizing a subject with one or more purified tumor antigens or one or more immunodominant VCX/Y-specific peptides (native or modified); alternately, antigen presenting cells pulsed with a VCX/Y-specific peptide (or transfected with genes encoding the tumor antigen) may be administered to a subject. The VCX/Y-specific peptide may be modified or contain one or more mutations such as, e.g., a substitution mutation, including a conservative mutation, for example. Passive immunotherapies include adoptive immunotherapies. Adoptive immunotherapies generally involve administering cells to a subject, wherein the cells (e.g., cytotoxic T cells) have been sensitized in vitro to the VCX/Y-specific peptide (see, e.g., U.S. Pat. No. 7,910,109).

In particular, a patient's own VCX/Y-specific T cells can be generated ex vivo for effective immune-based therapies within a short period of time, such as 6 to 8 weeks. The T cells may be isolated and expanded from autologous or allogeneic T cells (e.g., CD4⁺T cells, CD8⁺T cells, γδ T cells and/or Tregs) isolated from peripheral blood, such as with the tetramer guided sorting and rapid expansion protocol (REP). Next, the peptide or corresponding coded polynucleotides can be loaded to dendritic cells, LCL, PBMC, and/or artificial antigen presenting cells (aAPCs), and then co-cultured with the T cells by several rounds of stimulation to generate antigen-specific CTL cell lines or clones. Furthermore, with manipulation of immune modulating parameters, the effector function and long term persistence in vivo of these expanded antigen specific T cells can be enhanced. These autologous CTL cells can be used for adoptive immunotherapy for VCX/Y positive cancer patients. Further, other VCX/Y-specific cells that can be generated from the present disclosure include autologous or allogeneic NK cells, invariant NK cells, NKT cells, mesenchymal stem cells (MSCs), and/or induced pluripotent stem (iPS) cells. These cells may be isolated from blood, bone marrow, lymph, umbilical cord, and/or lymphoid organs.

### I. Definitions

In keeping with long-standing patent law convention, the words “a” and “an” when used in the present specification in concert with the word comprising, including the claims, denote “one or more.” As used in the specification and claims, the singular form “a”, “an”, and “the” include plural references unless the context clearly dictates otherwise. For example, the term “a nucleic acid” includes a plurality of nucleic acids, including mixtures thereof. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially” of indicates that the listed elements are required or mandatory, but that no other elements are optional and may or may not be present depending upon whether or not they affect the activity or action of the listed elements.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used to indicate that a value includes the inherent variation of error for the device, the method being employed to determine the value, or the variation that exists among the study subjects.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a T cell therapy and/or peptides.

“Subject” and “patient” and “individual” refer to either a human or non-human, such as primates, mammals, and vertebrates. In particular embodiments, the subject is a human of any gender or age or race.

The term “therapeutic benefit” or “therapeutically effective” as used throughout this application refers to anything that directly or indirectly promotes or enhances the well-being of the subject with respect to the medical treatment of this condition. This includes, but is not limited to, a reduction in the frequency or severity of one or more signs or symptoms of a disease. For example, treatment of cancer may involve, for example, a reduction in the size of one or more tumors, a reduction in the invasiveness of one or more tumors, reduction in the growth rate of the cancer, reduction of tumor load, or prevention of metastasis or expansion of metastasis. Treatment of cancer may also refer to prolonging survival of a subject with cancer.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “antibody” herein is used in the broadest sense and specifically covers monoclonal antibodies (including full length monoclonal antibodies), polyclonal antibodies, multi-specific antibodies (e.g., bispecific antibodies), and antibody fragments so long as they exhibit the desired biological activity.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The phrases “pharmaceutical or pharmacologically acceptable or pharmaceutically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject. In some embodiments, the dosage of antigen-specific T cell infusion may comprise about 100 million to about 30 billion cells, such as 10, 15, or 20 billion cells.

The term “immune checkpoint” refers to a molecule such as a protein in the immune system that provides signals to its components in order to balance immune reactions. Known immune checkpoint proteins comprise CTLA-4, PD1 and its ligands PD-Ll and PD-L2 and in addition LAG-3, BTLA, B7H3, B7H4, TIM3, KIR. The pathways involving LAG3, BTLA, B7H3, B7H4, TIM3, and KIR are recognized in the art to constitute immune checkpoint pathways similar to the CTLA-4 and PD-1 dependent pathways (see e.g. Pardoll, 2012; Mellman et al., 2011.

An “immune checkpoint inhibitor” refers to any compound inhibiting the function of an immune checkpoint protein. Inhibition includes reduction of function and full blockade. In particular the immune checkpoint protein is a human immune checkpoint protein. Thus the immune checkpoint protein inhibitor in particular is an inhibitor of a human immune checkpoint protein.

As used herein, a “protective immune response” refers to a response by the immune system of a mammalian host to a cancer. A protective immune response may provide a therapeutic effect for the treatment of a cancer, e.g., decreasing tumor size or increasing survival.

As used herein, the term “antigen” is a molecule capable of being bound by an antibody or T-cell receptor. An antigen may generally be used to induce a humoral immune response and/or a cellular immune response leading to the production of B and/or T lymphocytes.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3-zeta, a transmembrane domain, and one or more endodomains. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3ζ, FcR, CD27, CD28, CD137, DAP10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

A polynucleotide or polynucleotide region (or a polypeptide or polypeptide region) has a certain percentage (for example, 80%, 85%, 90%, or 95%) of “sequence identity” or “homology” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenB ank+EMBL+DDBJ+PDB+GenB ank CDS translations+SwissProtein+SPupdate+PIR.

### II. VCX/Y Peptides

Embodiments of the present disclosure concern tumor antigen-specific peptides, such as peptides from the VCX/Y tumor antigen. In particular embodiments, the tumor antigen-specific peptides have the amino acid sequence of a VCX/Y peptide (SEVEEPLSQ: SEQ ID NO:1). The tumor antigen-specific peptide may have an amino acid sequence with at least 80, 85, 90, 95, 96, 97, 98, 99, or 100 percent sequence identity with the peptide sequence of SEQ ID NO:1.

As used herein, the term “peptide” encompasses amino acid chains comprising 7-35 amino acids, including 8-35 amino acid residues, such as 8-25 amino acids, or 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids in length, or any range derivable therein. For example, a VCX/Y peptide of the present disclosure may, in some embodiments, comprise or consist of or consist essentially of the VCX118 peptide of SEQ ID NO:1. The peptide is an antigenic peptide, in particular embodiments, and as used herein an “antigenic peptide” is a peptide which, when introduced into a vertebrate, can stimulate the production of antibodies in the vertebrate, i.e., is antigenic, and wherein the antibody can selectively recognize and/or bind the antigenic peptide. An antigenic peptide may comprise an immunoreactive VCX/Y peptide, and may comprise additional sequences. The additional sequences may or may not be derived from a native antigen and may be heterologous, and such sequences may, but need not, be immunogenic. In certain embodiments, the VCX/Y peptide is 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids in length, or any range derivable therein. In specific embodiments, the tumor antigen-specific peptide (e.g., a VCX/Y peptide) is from 8 to 35 amino acids in length. In some embodiments, the tumor antigen-specific peptide (e.g., a VCX/Y peptide) is from 7-10, 8-10, 9-10, 7-9, 7-8, or 8-9 amino acids in length.

As would be appreciated by one of skill in the art, MHC molecules can bind peptides of varying sizes, but typically not full length proteins. While MHC class I molecules have been traditionally described to bind to peptides of 8-11 amino acids long, it has been shown that peptides 15 amino acids in length can bind to MHC class I molecules by bulging in the middle of the binding site or extending out of the MHC class I binding groove (Guo et al., 1992; Burrows et al., 2006; Samino et al., 2006; Stryhn et al., 2000; Collins et al., 1994; Blanchard and Shastri, 2008). Further, recent studies also demonstrated that longer peptides may be more efficiently endocytosed, processed, and presented by antigen-presenting cells (Zwaveling et al., 2002; Bijker et al., 2007; Melief and van der Burg, 2008; Quintarelli et al., 2011). As demonstrated in Zwaveling et al. (2002) peptides up to 35 amino acids in length may be used to selectively bind a class II MHC and are effective. As would be immediately appreciated by one of skill, a naturally occurring full-length tumor antigen, such as VCX/Y, would not be useful to selectively bind a class II MHC such that it would be endocytosed and generate proliferation of T cells. Generally, the naturally occurring full-length tumor antigen proteins do not display these properties and would thus not be useful for these immunotherapy purposes.

In certain embodiments, a tumor antigen-specific peptide (e.g., a VCX/Y peptide) is immunogenic or antigenic. As shown in the below examples, various tumor antigen-specific peptides (e.g., a VCX/Y peptide) of the present disclosure can promote the proliferation of T cells. It is anticipated that such peptides may be used to induce some degree of protective immunity.

A tumor antigen-specific peptide (e.g., a VCX/Y peptide) may be a recombinant peptide, synthetic peptide, purified peptide, immobilized peptide, detectably labeled peptide, encapsulated peptide, or a vector-expressed peptide (e.g., a peptide encoded by a nucleic acid in a vector comprising a heterologous promoter operably linked to the nucleic acid). In some embodiments, a synthetic tumor antigen-specific peptide (e.g., a VCX/Y peptide) may be administered to a subject, such as a human patient, to induce an immune response in the subject. Synthetic peptides may display certain advantages, such as a decreased risk of bacterial contamination, as compared to recombinantly expressed peptides. A tumor antigen-specific peptide (e.g., a VCX/Y peptide) may also be comprised in a pharmaceutical composition such as, e.g., a vaccine composition, which is formulated for administration to a mammalian or human subject.

### A. Cell Penetrating Peptides

In some embodiments, an immunotherapy may utilize a tumor antigen-specific peptide (e.g., a VCX/Y peptide) of the present disclosure that is associated with a cell penetrator, such as a liposome or a cell penetrating peptide (CPP). Antigen presenting cells (such as dendritic cells) pulsed with peptides may be used to enhance antitumor immunity (Celluzzi et al., 1996; Young et al., 1996). Liposomes and CPPs are described in further detail below. In some embodiments, an immunotherapy may utilize a nucleic acid encoding a tumor antigen-specific peptide (e.g., a VCX/Y peptide) of the present disclosure, wherein the nucleic acid is delivered, e.g., in a viral vector or non-viral vector.

A tumor antigen-specific peptide (e.g., a VCX/Y peptide) may also be associated with or covalently bound to a cell penetrating peptide (CPP). Cell penetrating peptides that may be covalently bound to a tumor antigen-specific peptide (e.g., a VCX/Y peptide) include, e.g., HIV Tat, herpes virus VP22, the Drosophila Antennapedia homeobox gene product, signal sequences, fusion sequences, or protegrin I. Covalently binding a peptide to a CPP can prolong the presentation of a peptide by dendritic cells, thus enhancing antitumour immunity (Wang and Wang, 2002). In some embodiments, a tumor antigen-specific peptide (e.g., the VCX/Y peptide) of the present disclosure (e.g., comprised within a peptide or polyepitope string) may be covalently bound (e.g., via a peptide bond) to a CPP to generate a fusion protein. In other embodiments, a tumor antigen-specific peptide (e.g., a VCX/Y peptide) or nucleic acid encoding a tumor antigen-specific peptide may be encapsulated within or associated with a liposome, such as a mulitlamellar, vesicular, or multivesicular liposome, an exocytic vesicle or exosome.

As used herein, “association” means a physical association, a chemical association or both. For example, an association can involve a covalent bond, a hydrophobic interaction, encapsulation, surface adsorption, or the like.

As used herein, “cell penetrator” refers to a composition or compound that enhances the intracellular delivery of the peptide/polyepitope string to the antigen presenting cell. For example, the cell penetrator may be a lipid which, when associated with the peptide, enhances its capacity to cross the plasma membrane. Alternatively, the cell penetrator may be a peptide. Cell penetrating peptides (CPPs) are known in the art, and include, e.g., the Tat protein of HIV (Frankel and Pabo, 1988), the VP22 protein of HSV (Elliott and O′Hare, 1997) and fibroblast growth factor (Lin et al., 1995).

Cell-penetrating peptides (or “protein transduction domains”) have been identified from the third helix of the Drosophila Antennapedia homeobox gene (Antp), the HIV Tat, and the herpes virus VP22, all of which contain positively charged domains enriched for arginine and lysine residues (Schwarze et al., 2000; Schwarze et al., 1999). Also, hydrophobic peptides derived from signal sequences have been identified as cell-penetrating peptides. (Rojas et al., 1996; Rojas et al., 1998; Du et al., 1998). Coupling these peptides to marker proteins such as β-galactosidase has been shown to confer efficient internalization of the marker protein into cells, and chimeric, in-frame fusion proteins containing these peptides have been used to deliver proteins to a wide spectrum of cell types both in vitro and in vivo (Drin et al., 2002). Fusion of these cell penetrating peptides to a tumor antigen-specific peptide (e.g., a VCX/Y peptide) in accordance with the present disclosure may enhance cellular uptake of the polypeptides.

In some embodiments, cellular uptake is facilitated by the attachment of a lipid, such as stearate or myristilate, to the polypeptide. Lipidation has been shown to enhance the passage of peptides into cells. The attachment of a lipid moiety is another way that the present disclosure increases polypeptide uptake by the cell. Cellular uptake is further discussed below.

A tumor antigen-specific peptide (e.g., a VCX/Y peptide) of the present disclosure may be included in a liposomal vaccine composition. For example, the liposomal composition may be or comprise a proteoliposomal composition. Methods for producing proteoliposomal compositions that may be used with the present disclosure are described, e.g., in Neelapu et al. (2007) and Popescu et al. (2007). In some embodiments, proteoliposomal compositions may be used to treat a melanoma.

By enhancing the uptake of a tumor antigen-specific polypeptide, it may be possible to reduce the amount of protein or peptide required for treatment. This in turn can significantly reduce the cost of treatment and increase the supply of therapeutic agent. Lower dosages can also minimize the potential immunogencity of peptides and limit toxic side effects.

In some embodiments, a tumor antigen-specific peptide (e.g., a VCX/Y peptide) may be associated with a nanoparticle to form nanoparticle-polypeptide complex. In some embodiments, the nanoparticle is a liposomes or other lipid-based nanoparticle such as a lipid-based vesicle (e.g., a DOTAP:cholesterol vesicle). In other embodiments, the nanoparticle is an iron-oxide based superparamagnetic nanoparticles. Superparamagnetic nanoparticles ranging in diameter from about 10 to 100 nm are small enough to avoid sequestering by the spleen, but large enough to avoid clearance by the liver. Particles this size can penetrate very small capillaries and can be effectively distributed in body tissues. Superparamagnetic nanoparticles-polypeptide complexes can be used as MRI contrast agents to identify and follow those cells that take up the tumor antigen-specific peptide (e.g., a VCX/Y peptide). In some embodiments, the nanoparticle is a semiconductor nanocrystal or a semiconductor quantum dot, both of which can be used in optical imaging. In further embodiments, the nanoparticle can be a nanoshell, which comprises a gold layer over a core of silica. One advantage of nanoshells is that polypeptides can be conjugated to the gold layer using standard chemistry. In other embodiments, the nanoparticle can be a fullerene or a nanotube (Gupta et al., 2005).

Peptides are rapidly removed from the circulation by the kidney and are sensitive to degradation by proteases in serum. By associating a tumor antigen-specific peptide (e.g., a VCX/Y peptide) with a nanoparticle, the nanoparticle-polypeptide complexes of the present disclosure may protect against degradation and/or reduce clearance by the kidney. This may increase the serum half-life of polypeptides, thereby reducing the polypeptide dose need for effective therapy. Further, this may decrease the costs of treatment, and minimizes immunological problems and toxic reactions of therapy.

### B. Polyepitope Strings

In some embodiments, a tumor antigen-specific peptide (e.g., a VCX/Y peptide) is included or comprised in a polyepitope string. A polyepitope string is a peptide or polypeptide containing a plurality of antigenic epitopes from one or more antigens linked together. A polyepitope string may be used to induce an immune response in a subject, such as a human subject. Polyepitope strings have been previously used to target malaria and other pathogens (Baraldo et al., 2005; Moorthy et al., 2004; Baird et al., 2004). A polyepitope string may refer to a nucleic acid (e.g., a nucleic acid encoding a plurality of antigens including a VCX/Y peptide) or a peptide or polypeptide (e.g., containing a plurality of antigens including a VCX/Y peptide). A polyepitope string may be included in a cancer vaccine composition.

### C. Biological Functional Equivalents

A tumor antigen-specific peptide (e.g., a VCX/Y peptide) of the present disclosure may be modified to contain amino acid substitutions, insertions and/or deletions that do not alter their respective interactions. Such a biologically functional equivalent of a tumor antigen-specific peptide (e.g., a VCX/Y peptide) could be a molecule having like or otherwise desirable characteristics. As a non-limiting example, certain amino acids may be substituted for other amino acids in a tumor antigen-specific peptide (e.g., a VCX/Y peptide) disclosed herein without appreciable loss of interactive capacity. In some embodiments, the tumor antigen-specific peptide has a substitution mutation at an anchor reside, such as a substitution mutation at one, two, or all of positions: 1 (P1), 2 (P2), and/or 9 (P9). It is thus contemplated that a tumor antigen-specific peptide (e.g., a VCX/Y peptide) disclosed herein (or a nucleic acid encoding such a peptide) which is modified in sequence and/or structure, but which is unchanged in biological utility or activity remains within the scope of the compositions and methods disclosed herein.

It is also well understood by the skilled artisan that, inherent in the definition of a biologically functional equivalent peptide, is the concept that there is a limit to the number of changes that may be made within a defined portion of the molecule while still maintaining an acceptable level of equivalent biological activity. Biologically functional equivalent peptides are thus defined herein as those peptides in which certain, not most or all, of the amino acids may be substituted. Of course, a plurality of distinct peptides with different substitutions may easily be made and used in accordance with the present disclosure.

The skilled artisan is also aware that where certain residues are shown to be particularly important to the biological or structural properties of a peptide, e.g., residues in specific epitopes, such residues may not generally be exchanged. This may be the case in the present disclosure, as a mutation in an tumor antigen-specific peptide (e.g., the VCX/Y peptide) disclosed herein could result in a loss of species-specificity and in turn, reduce the utility of the resulting peptide for use in methods of the present disclosure. Thus, peptides which are antigenic and comprise conservative amino acid substitutions are understood to be included in the present disclosure. Conservative substitutions are least likely to drastically alter the activity of a protein. A “conservative amino acid substitution” refers to replacement of amino acid with a chemically similar amino acid, i.e., replacing nonpolar amino acids with other nonpolar amino acids; substitution of polar amino acids with other polar amino acids, acidic residues with other acidic amino acids, etc.

Amino acid substitutions, such as those which might be employed in modifying a tumor antigen-specific peptide (e.g., a VCX/Y peptide) disclosed herein are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. In some embodiments, the mutation may enhance TCR-pMHC interaction and/or peptide-MHC binding.

The present disclosure also contemplates isoforms of the tumor antigen-specific peptides (e.g., a VCX/Y peptide) disclosed herein. An isoform contains the same number and kinds of amino acids as a peptide of the present disclosure, but the isoform has a different molecular structure. The isoforms contemplated by the present disclosure are those having the same properties as a peptide of the present disclosure as described herein.

Nonstandard amino acids may be incorporated into proteins by chemical modification of existing amino acids or by de novo synthesis of a peptide disclosed herein. A nonstandard amino acid refers to an amino acid that differs in chemical structure from the twenty standard amino acids encoded by the genetic code.

In select embodiments, the present disclosure contemplates a chemical derivative of a tumor antigen-specific peptide (e.g., a VCX/Y peptide) disclosed herein. “Chemical derivative” refers to a peptide having one or more residues chemically derivatized by reaction of a functional side group, and retaining biological activity and utility. Such derivatized peptides include, for example, those in which free amino groups have been derivatized to form specific salts or derivatized by alkylation and/or acylation, p-toluene sulfonyl groups, carbobenzoxy groups, t-butylocycarbonyl groups, chloroacetyl groups, formyl or acetyl groups among others. Free carboxyl groups may be derivatized to form organic or inorganic salts, methyl and ethyl esters or other types of esters or hydrazides and preferably amides (primary or secondary). Chemical derivatives may include those peptides which comprise one or more naturally occurring amino acids derivatives of the twenty standard amino acids. For example, 4-hydroxyproline may be substituted for serine; and ornithine may be substituted for lysine.

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The amino acids described herein are preferred to be in the “L” isomeric form. However, residues in the “D” isomeric form can be substituted for any L-amino acid residue, as long as the desired functional properties set forth herein are retained by the protein.

### D. Nucleic Acids Encoding a Tumor Antigen-Specific Peptide

In an aspect, the present disclosure provides a nucleic acid encoding an isolated antigen-specific peptide comprising a sequence that has at least about 90%, 91%, 92%, 93%, 94%, 95%, 96%, 97%, 98%, 99%, or 100% sequence identity SEQ ID NO:1, or the peptide may have 1, 2, 3, or 4 point mutations (e.g., substitution mutations) as compared to SEQ ID NO:1. As stated above, such a tumor antigen-specific peptide may be, e.g., from 8 to 35 amino acids in length, or any range derivable therein. In some embodiments, the tumor antigen-specific peptide corresponds to a portion of the tumor antigen protein such as VCX1, VCX2, VCX3A, VCX3B, or VCY (e.g., VCX3A; GenBank Accession No: AAI26903.1). The term “nucleic acid” is intended to include DNA and RNA and can be either double stranded or single stranded.

Some embodiments of the present disclosure provide recombinantly-produced tumor antigen-specific peptides (e.g., a VCX/Y peptide). Accordingly, a nucleic acid encoding a tumor antigen-specific peptide may be operably linked to an expression vector and the peptide produced in the appropriate expression system using methods well known in the molecular biological arts. A nucleic acid encoding a tumor antigen-specific peptide disclosed herein may be incorporated into any expression vector which ensures good expression of the peptide. Possible expression vectors include but are not limited to cosmids, plasmids, or modified viruses (e.g. replication defective retroviruses, adenoviruses and adeno-associated viruses), so long as the vector is suitable for transformation of a host cell.

A recombinant expression vector being “suitable for transformation of a host cell” means that the expression vector contains a nucleic acid molecule of the present disclosure and regulatory sequences selected on the basis of the host cells to be used for expression, which is operatively linked to the nucleic acid molecule. The terms, “operatively linked” or “operably linked” are used interchangeably, and are intended to mean that the nucleic acid is linked to regulatory sequences in a manner which allows expression of the nucleic acid.

Accordingly, the present disclosure provides a recombinant expression vector comprising nucleic acid encoding a tumor antigen-specific peptide, and the necessary regulatory sequences for the transcription and translation of the inserted protein-sequence. Suitable regulatory sequences may be derived from a variety of sources, including bacterial, fungal, or viral genes (e.g., see the regulatory sequences described in Goeddel (1990).

Selection of appropriate regulatory sequences is generally dependent on the host cell chosen, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native protein and/or its flanking regions.

A recombinant expression vector may also contain a selectable marker gene which facilitates the selection of host cells transformed or transfected with a recombinant tumor antigen-specific peptides (e.g., a VCX/Y peptide) disclosed herein. Examples of selectable marker genes are genes encoding a protein such as G418 and hygromycin which confer resistance to certain drugs, β-galactosidase, chloramphenicol acetyltransferase, or firefly luciferase. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of a recombinant expression vector, and in particular, to determine the effect of a mutation on expression and phenotype. It will be appreciated that selectable markers can be introduced on a separate vector from the nucleic acid of interest.

Recombinant expression vectors can be introduced into host cells to produce a transformant host cell. The term “transformant host cell” is intended to include prokaryotic and eukaryotic cells which have been transformed or transfected with a recombinant expression vector of the present disclosure. The terms “transformed with”, “transfected with”, “transformation” and “transfection” are intended to encompass introduction of nucleic acid (e.g. a vector) into a cell by one of many possible techniques known in the art. Suitable host cells include a wide variety of prokaryotic and eukaryotic host cells. For example, the proteins of the present disclosure may be expressed in bacterial cells such as E. coli, insect cells (using baculovirus), yeast cells or mammalian cells.

A nucleic acid molecule of the present disclosure may also be chemically synthesized using standard techniques. Various methods of chemically synthesizing polydeoxy-nucleotides are known, including solid-phase synthesis which, like peptide synthesis, has been fully automated in commercially available DNA synthesizers (See e.g., U.S. Pat. Nos. 4,598,049; 4,458,066; 4,401,796; and 4,373,071).

### III. Antigen-Specific Cell Therapy

Embodiments of the present disclosure concern (optionally obtaining and) administering antigen-specific cells (e.g., autologous or allogeneic T cells (e.g., regulatory T cells, CD4⁺T cells, CD8⁺T cells, or gamma-delta T cells), NK cells, invariant NK cells, NKT cells, mesenchymal stem cell (MSC)s, and/or induced pluripotent stem (iPS) cells) to a subject as an immunotherapy to target cancer cells. In particular, the cells are antigen-specific T cells (e.g., VCX/Y-specific T cells). Several basic approaches for the derivation, activation and expansion of functional anti-tumor effector cells have been described in the last two decades. These include the following: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization that can be used in the methods described herein.

In some embodiments, the T cells are derived from the blood, bone marrow, lymph, umbilical cord, and/or lymphoid organs. In some aspects, the cells are human cells. The cells typically are primary cells, such as those isolated directly from a subject and/or isolated from a subject and frozen. They may or may not be isolated from an individual in need of the therapy of the disclosure. In some embodiments, the cells include one or more subsets of T cells or other cell types, such as whole T cell populations, CD4⁺cells, CD8⁺cells, and subpopulations thereof, such as those defined by function, activation state, maturity, potential for differentiation, expansion, recirculation, localization, and/or persistence capacities, antigen- specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same individual, before or after cryopreservation.

Among the sub-types and subpopulations of T cells (e.g., CD4⁺and/or CD8⁺T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and/or [NRF1]delta/gamma T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD4⁺or CD8⁺selection step is used to separate CD4⁺helper and CD8⁺cytotoxic T cells. Such CD4⁺and CD8⁺populations can be further sorted into sub-populations by positive or negative selection for markers expressed or expressed to a relatively higher degree on one or more naive, memory, and/or effector T cell subpopulations.

In some embodiments, CD8⁺T cells are further enriched for or depleted of naive, central memory, effector memory, and/or central memory stem cells, such as by positive or negative selection based on one or more surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al., 2012; Wang et al., 2012.

In some embodiments, the T cells are autologous T cells. In this method, tumor samples are obtained from individuals, including patients, and a single cell suspension is obtained. The single cell suspension can be obtained in any suitable manner, e.g., mechanically (disaggregating the tumor using, e.g., a gentleMACSTM Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T cell receptor stimulation in the presence of feeder lymphocytes and interleukin-2 (IL-2) and/or interleukin-15 (IL-15). In some cases, the non-specific T cell receptor stimulus can include an anti-CD3 antibody, such as around 30 ng/ml of OKT3, a mouse monoclonal anti-CD3 antibody (available from Ortho-McNeil®, Raritan, N.J.). Alternatively, T cells can be rapidly expanded by stimulation of PBMCs in vitro with one or more antigens (including antigenic portions thereof, such as epitope(s), or a cell) of the cancer, which can be optionally expressed from a vector, such a tumor peptide, in the presence of a T cell growth factor, such as 300 IU/ml IL-2 and/or IL-15. The in vitro-induced T cells are rapidly expanded by re-stimulation with the same antigen(s) of the cancer pulsed onto antigen-presenting cells. Alternatively, the T cells can be re-stimulated with irradiated, autologous lymphocytes or with irradiated allogeneic lymphocytes and IL-2, for example.

The autologous T cells can be modified to express a T cell growth factor that promotes the growth and activation of the autologous T cells. Suitable T cell growth factors include, for example, IL-2, IL-7, IL-15, and/or IL-12. Suitable methods of modification are known in the art. See, for instance, Sambrook et al., 2001; and Ausubel et al., 1994. In particular aspects, modified autologous T cells express the T cell growth factor at high levels. T cell growth factor coding sequences, such as that of IL-12, are readily available in the art, as are promoters, the operable linkage of which to a T cell growth factor coding sequence promotes high-level expression.

### IV. Methods of Treatment

Provided herein are methods for treating or delaying progression of cancer in an individual comprising administering to the individual an effective amount an antigen-specific cell therapy, such as a VCX/Y-specific T cell therapy. In further embodiments, methods are provided for the treatment of cancer comprising immunizing a subject with a purified tumor antigen or an immunodominant tumor antigen-specific peptide.

The VCX/Y peptide provided herein can be utilized to develop cancer vaccines or immunogens (e.g., a peptide or modified peptide mix with adjuvant, coding polynucleotide and corresponding expression products such as inactive virus or other microorganisms vaccine). These peptide specific vaccines or immunogens can be used for immunizing cancer patients directly to induce anti-tumor immuno-response in vivo, or for expanding antigen specific T cells in vitro with peptide or coded polynucleotide loaded APC stimulation. These large number of T cells can be adoptively transferred to patients to induce tumor regression.

Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

In some embodiments, T cells are autologous. However the cells can be allogeneic. In some embodiments, the T cells are isolated from the individual in need of treatment, so that the cells are autologous. If the T cells are allogeneic, the T cells can be pooled from several donors. The cells are administered to the subject of interest in an amount sufficient to control, reduce, or eliminate symptoms and signs of the disease being treated.

In some embodiments, the subject can be administered nonmyeloablative lymphodepleting chemotherapy prior to the T cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a T-cell growth factor that promotes the growth and activation of the autologous T cells is administered to the subject either concomitantly with the autologous T cells or subsequently to the autologous T cells. The T-cell growth factor can be any suitable growth factor that promotes the growth and activation of the autologous T- cells. Examples of suitable T-cell growth factors include IL-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 may be utilized, in particular embodiments.

The T cell may be administered intravenously, intramuscularly, subcutaneously, topically, orally, transdermally, intraperitoneally, intraorbitally, by implantation, by inhalation, intrathecally, intraventricularly, or intranasally. The appropriate dosage of the T cell therapy may be determined based on the type of disease to be treated, severity and course of the disease, the clinical condition of the individual, the individual's clinical history and response to the treatment, and the discretion of the attending physician.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

### A. Pharmaceutical Compositions

Also provided herein are pharmaceutical compositions and formulations comprising antigen-specific immune cells (e.g., T cells) and a pharmaceutically acceptable carrier. A vaccine composition for pharmaceutical use in a subject may comprise a tumor antigen peptide (e.g., VCX/Y) composition disclosed herein and a pharmaceutically acceptable carrier. An individual may receive an effective amount of one or both.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn- protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. publication Nos. 2005/0260186 and 2006/0104968. In one aspect, a sHASEGP is combined with one or more additional glycosaminoglycanases such as chondroitinases.

### B. Combination Therapies

In certain embodiments, the compositions and methods of the present embodiments involve an antigen-specific immune cell population and/or tumor antigen peptides in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side- effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an antigen-specific immune cell therapy, or peptide is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody—drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication W02015016718; Pardo11, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP- 224. Nivolumab, also known as MDX-1106-04, MDX-1106, ONO-4538, BMS-936558, and OPDIVO®, is an anti- PD-1 antibody described in W02006/121168. Pembrolizumab, also known as MK-3475, Merck 3475, lambrolizumab, KEYTRUDA®, and SCH-900475, is an anti-PD-1 antibody described in W02009/114335. CT-011, also known as hBAT or hBAT-1, is an anti-PD-1 antibody described in W02009/101611. AMP-224, also known as B7-DCIg, is a PDL2-Fc fusion soluble receptor described in W02010/027827 and W02011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Patent No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX- 010, MDX- 101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above- mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. US5844905, US5885796 and International Patent Application Nos. WO1995001994 and WO1998042752; all incorporated herein by reference, and immunoadhesins such as described in U.S. Pat. No. US8329867, incorporated herein by reference.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. Articles of Manufacture or Kits

An article of manufacture or a kit is provided comprising antigen-specific immune cells, TCRs, and/or antigen peptides (e.g., VCX/Y peptide). The article of manufacture or kit can further comprise a package insert comprising instructions for using the antigen-specific immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Identification and Characterization of Tumor Antigen-Specific Peptides

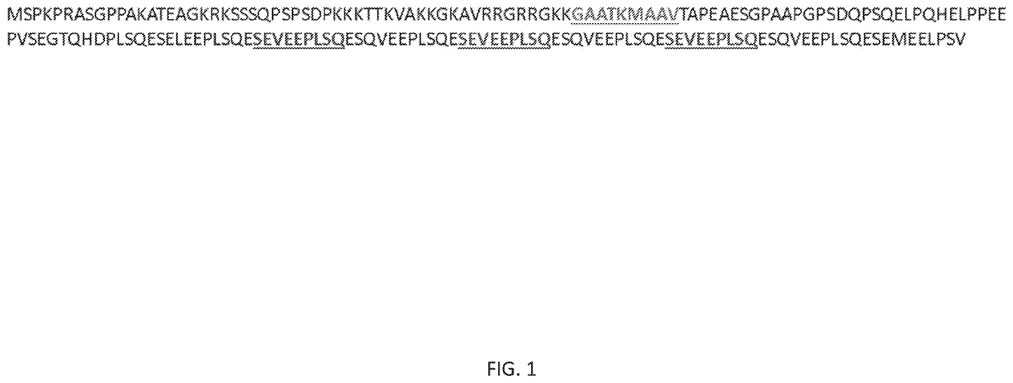

Overlap peptide library screening was performed for the recognition target peptide of VCX54 TCR-T cells (FIG. 1). The 9-mer peptide with 8 amino acid overlap peptide library covering the whole length of VCX3A protein was screened for the VCX54 TCR-T cell recognized target. The peptide was pulsed to T2 cell one by one and co-cultured with VCX54 TCR-T. After overnight culturing, the activated marker of T cells such as CD137, CD69, IFN-r and TNFa were detected using intracellular cytokine staining (ICS). The target peptide, VCX54, was found to be able to induce the response of VCX54 TCR-T. However, anther peptide, VCX118, was found to also be able to induce the response of VCX54 TCR-T cells.

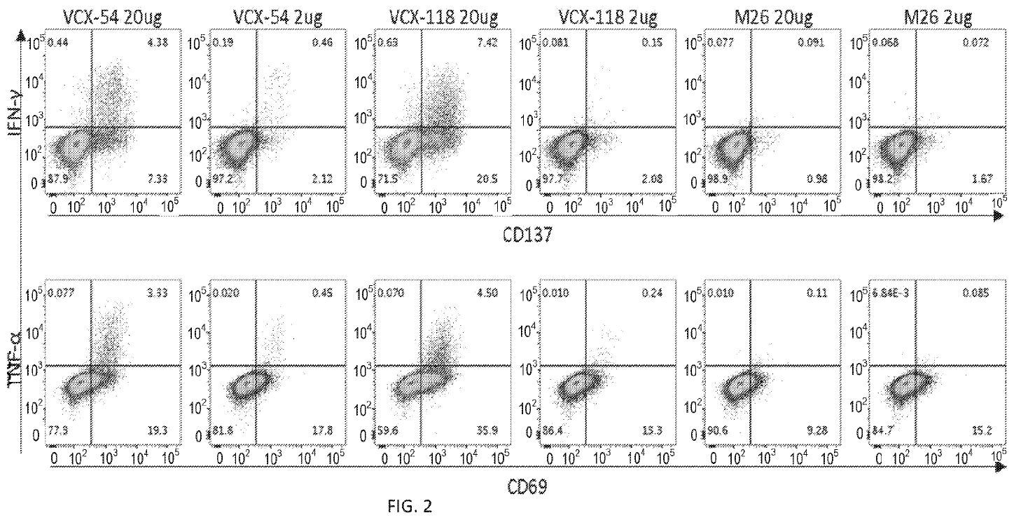

For VCX118 peptide cross reaction detection, different concentration of VCX118 peptide were used to pulse T2 cells and then co-cultured with VCX54 TCR-T cells. The ICS detection show at high concentration, the VCX118 peptide will be cross recognized by VCX54 TCR-T, and the response was higher than the VCX54 original peptide. M26 peptide was used as a negative control (FIG. 2).

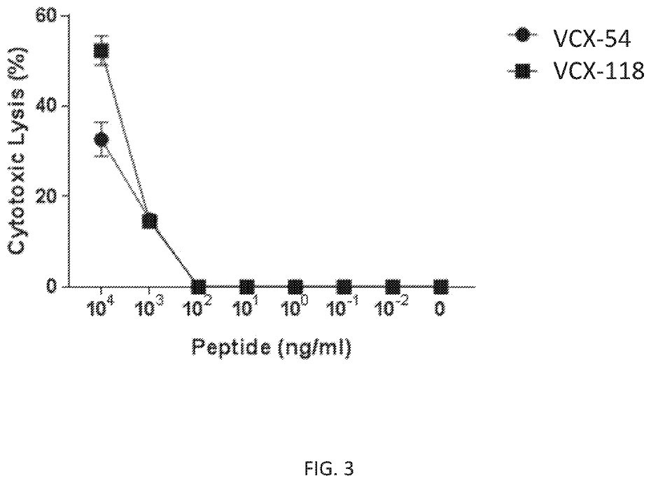

Different concentrations of VCX118 peptide were pulsed to T2 cells, labeled with Calcein-AM, and co-cultured with VCX54 TCR-T cells. The calcein-AM quenching assay was used to detect VCX54 TCR-T killing ability in response to the antigen. The killing assay showed that at high concentrations, VCX54 TCR-T cells can cross recognize the VCX118 peptide and the response is higher than the VCX54 peptide (FIG. 3).

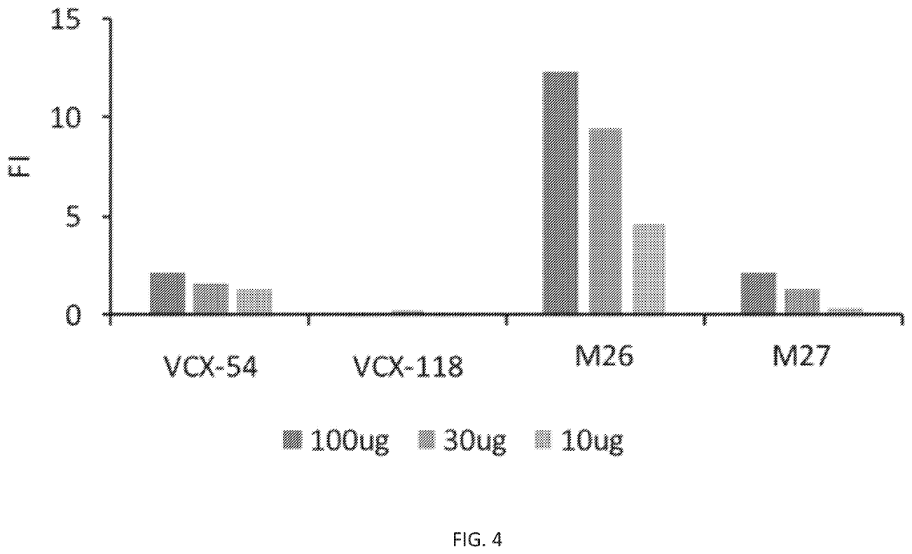

Different concentrations of VCX118 peptide were pulsed to T2 cells, and the HLA-A2 stability analysis was performed to detect the VCX118 peptide binding ability. VCX54, M26 and M27 peptide were used as control. From the analysis, VCX118 showed weak binding ability to HLA-A2 (FIG. 4).

Table 1
Different lengths of peptide comprising the
VCX118 peptide.

| | | NetMHC | |
|---|---|---|---|
| | | Binding | |
| Length | Sequence | (nM) | |
| 9 | SEVEEPLSQ (SEQ ID | 754101.8 | VCX-118-126 |
| | NO: 1) | | |
| 10 | ESEVEEPLSQ (SEQ ID | 421521.8 | VCX-117-126 |
| | NO: 2) | | |
| 10 | SEVEEPLSQE (SEQ ID | 47186.7 | VCX-118-127 |
| | NO: 3) | | |
| 11 | QESEVEEPLSQ (SEQ ID | 13777.8 | VCX-116-126 |
| | NO: 4) | | |
| 11 | ESEVEEPLSQE (SEQ ID | 94881.2 | VCX-117-127 |
| | NO: 5) | | |
| 11 | SEVEEPLSQES (SEQ ID | 635.6 | VCX-118-128 |
| | NO: 6) | | |

Different length of peptides with VCX118 were pulsed to T2 cells and co-cultured with VCX54 TCR-T cells. The VCX118 peptide was used as a control. The ICS data showed that VCX54 TCR-T cells only recognized the VCX118 peptide but not other longer peptides, indicating that VCX54 TCR-T cells might only cross recognize VCX118 peptide in the VCX3A gene.

### Example 2—Materials and Methods

Generation and expansion of VCX118-specific CD8 T cells: Tumor antigen—specific CTLs were generated with a manner previously described (Li 2005). Leukapheresis PBMCs were stimulated by autologous DC pulsed with tumor antigen peptide. For induction of dendritic cell, adherent PBMCs were cultured with GM-CSF and IL-4 in AIM-V medium (Invitrogen Life Technologies) for 6 days and then added IL-1β, IL-6, TNF-αand PGE2 for maturation. After 1 day, mature DCs were pulsed with 40 μg/ml peptide at 2×10⁶ cells/ml of 1% human serum albumin (HSA)/PBS in the present of 3 μg/ml beta-microglobulin for 4hr at room temperature. After washing with 1% HSA/PBS, DCs were mixed with PBMCs at 1.5×10⁶ cell /ml/well in 48 well plate. IL-21 (30 ng/ml) was added initially and 3-4 days after culture. IL-2 and IL-7 were added 1 day after secondary stimulation to expand activated antigen—specific T cells.

6 days after secondary stimulation, cells were stained with VCX/Y peptide/MHC—PE-conjugated tetramer and CD8—APC antibody, and then CD8 and tetramer-positive cells were sorted by ARIA II. The sorted VCX/Y-specific CD8 T cells were expanded by Rapid Expansion Protocol (REP) with feeder cells of PBL and LCL under IL-21.

Peptide—MHC tetramer staining: VCX118—specific CD8 T cells were confirmed by staining with tetramer of VCX118 peptide/MHC complex. CD8 T cells were incubated with PE-conjugated tetramer for 20 mins, washed and then stained with APC-conjugated CD8 antibody for 15 mins in room temperature. After washing, cells were analyzed by flow cytometry (LSRFortessa X-20 Analyzer).

Generate T cell clone: The whole length VCX3A RNA was transfected to matured dendritic cells (DC). The RNA transfected DC were co-cultured with autogenetic naïve T cell at the ratio of DC: T=1:10 in the presence of IL-21. After one week, the RNA-transfected DC were used to stimulate the T cells again. After two round of stimulation, the CD8+ and tetramer+ double positive T cell population were sorted and expanded with rapid expansion protocol. The T cell clones were generated with limiting dilution method. The high activity CTL clones were screened via tumor cells killing assay.

⁵¹Cr release assay: The killing ability of the T cell or CTL clone to lyse tumor targets was measured using a standard ⁵¹Cr release assay. Tumor cells or normal cells were labeled for 2 h at 37° C. with 200 μCi of ⁵¹Cr. Labeled target cells were washed and then incubated with effector cells at the different ratios for 4 h at 37° C. in 0.2 ml of complete medium. Harvested supernatants were counted using automatic gamma counter. Maximal and spontaneous ⁵¹Cr release was determined by incubating the labeled target cells in either trypan lysis buffer or medium for 4 h at 37° C. Each data point was determined as an average of quadruplicate wells. The percent specific lysis was calculated as follows: % killing=((specific release —spontaneous release)/(total release - spontaneous release))×100.

IFN-γ release assay: IFN-γ release from T cell was detected with ELISA method. The T cells were incubated with target cells at 10:1 ration in 96 well plate with 0.2 ml medium at 37° C. After co-culturing overnight, the supernatant was harvested and the IFN-y concentration was detected using ELISA according to the manual of the kit (Invitrogen Life Technologies).

Intracellular cytokine staining (ICS) assay: The T cells were incubated with target cells at 10:1 ration in the presence of brefeldin A (BFA) at 37° C. overnight. After co-culturing, the T cells were harvested and washed. The cells were stained with flow antibody anti surface marker first. After that, the cells were washed and fixed with Fix Buffer and then were permeabilized using Permeabilizing Solution (eBioscience). Permeabilized cells are then stained with intracellular cytokine flow antibody. Finally, the level of cytokine producing in the cells was analyzed using FACS.

Statistical analysis: Data analysis was performed using GraphPad prism version 6.0e. Normally distributed data were analyzed using parametric tests (Anova or unpaired t-test). Statistical test differences were considered significant if p values were <0.05.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Austin-Ward and Villaseca, Revista Medica de Chile, 126(7):838-845, 1998.
Ausubel et al., Current Protocols in Molecular Biology, Greene Publishing Associates and John
Baird et al., Scand. J. Immunol., 60(4):363-71, 2004.
Baraldo et al., Infect. Immun., 73(9):5835-41, 2005.
Bijker et al., J. Immunol., 179:5033-5040, 2007. Biology Publications, p. 433, 1997.
Blanchard and Shastri, Curr. Opin. Immunol., 20:82-88, 2008.
Bukowski et al., Clinical Cancer Res., 4(10):2337-2347, 1998.
Burrows et al., Trends Immunol., 27:11-16, 2006.
Camacho et al. (2004) J Clin Oncology 22(145): Abstract No. 2505, 2004.
Celluzzi et al., J. Exp. Med., 183 283-287, 1996.
Chothia et al., EMBO J. 7:3745, 1988.
Christodoulides et al., Microbiology, 144(Pt 11):3027-3037, 1998.
Cohen et al. J Immunol. 175:5799-5808, 2005. Cold Spring Harbor, N.Y. 2001.
Collins et al., Nature, 371:626-629, 1994.
Davidson et al., J. Immunother., 21(5):389-398, 1998.
Davila et al. PLoS ONE 8(4): e61338, 2013.
Drin et al., AAPS Pharm. Sci., 4(4):E26, 2002.
Du et al., J. Pept. Res., 51:235-243, 1998.
Dudley et al., J. Immunol., 26(4):332-342, 2003.
Elliott and O′Hare, Cell, 88:23-233, 1997.
European Patent Application No. EP2537416
Fedorov et al., Sci. Transl. Medicine, 5(215) 2013.
Janeway et al, Immunobiology: The Immune System in Health and Disease, 3^{rd} Ed., Current
Frankel and Pabo, Cell, 55:189-1193, 1988.
Goeddel, Methods Enzymol., 185:3-7, 1990.
Guo et al., Nature, 360:364-366, 1992.
Gupta et al., Biomaterials, 26:3995-4021, 2005.
Hanibuchi et al., Int. J. Cancer, 78(4):480-485, 1998.
Heemskerk et al. Hum Gene Ther. 19:496-510, 2008.
Hellstrand et al., Acta Oncologica, 37(4):347-353, 1998.
Hollander, Front. Immun., 3:3, 2012.
Hui and Hashimoto, Infection Immun., 66(11):5329-5336, 1998.
Hurwitz et al. (1998) Proc Natl Acad Sci USA 95(17): 10067-10071
International Patent Publication No. WO 00/37504
International Patent Publication No. WO 01/14424
International Patent Publication No. WO 98/42752
International Patent Publication No. WO 99/60120
International Patent Publication No. W01995001994
International Patent Publication No. W01998042752
International Patent Publication No. W02000037504
International Patent Publication No. W0200014257
International Patent Publication No. W02001014424
International Patent Publication No. W02006/121168
International Patent Publication No. W02007/103009
International Patent Publication No. W02009/101611
International Patent Publication No. W02010/027827
International Patent Publication No. W02011/066342
International Patent Publication No. W02012/129514
International Patent Publication No. W02013/071154
International Patent Publication No. W02013/123061
International Patent Publication No. W02013/166321
International Patent Publication No. W02013126726
International Patent Publication No. W02014/055668
International Patent Publication No. W02014031687
International Patent Publication No. W02015016718
Janeway et al, Immunobiology: The Immune System in Health and Disease, 3^{rd} Ed., Current Biology Publications, p. 433, 1997.
Johnson et al. Blood 114:535-46, 2009.
Jores et al., PNAS U.S.A. 87:9138, 1990.
Kabat et al., “Sequences of Proteins of Immunological Interest, US Dept. Health and Human Services, Public Health Service National Institutes of Health, 5^{th} ed, 1991.
Leal, M., Ann N Y Acad Sci 1321, 41-54, 2014.
Lefranc et al., Dev. Comp. Immunol. 27:55, 2003.
Li, Nat Biotechnol. 23:349-354, 2005.
Lin et al., J. Biol. Chem., 270:4255-14258, 1995.
Melief and van der Burg, Nat. Rev. Cancer, 8:351-360, 2008.
Mellman et al., Nature 480:480- 489, 2011.
Mokyr et al. Cancer Res 58:5301-5304, 1998.
Moorthy et al., PLoS Med., 1(2):e33, 2004.
Neelapu et al., Blood, 15:109(12):5160-5163, 2007.
Pardoll, Nature Rev Cancer 12:252-264, 2012.
Parkhurst et al. Clin Cancer Res. 15: 169-180, 2009.
Popescu et al. Blood, 15:109(12):5407-5410, 2007.
Qin et al., Proc. Natl. Acad. Sci. USA, 95(24):14411-14416, 1998.
Quintarelli et al., Blood, 117:3353-3362, 2011.
Rojas et al., J. Biol. Chem., 271:27456-27461, 1996.
Rojas et al., Proc. West. Pharmacol. Soc., 41:55-56, 1998.
Sadelain et al., Cancer Discov. 3(4): 388-398, 2013.
Sambrook et al., Molecular Cloning: A Laboratory Manual, 3r^{d} ed., Cold Spring Harbor Press, 2001.
Samino et al., J. Biol. Chem., 281:6358-6365, 2006.
Schwarze et al., Trends in Cell Biol., 10:290-295, 2000.
Stryhn et al., Eur. J. Immunol., 30:3089-3099, 2000.
Terakura et al. Blood.1:72- 82, 2012.
Turtle et al., Curr. Opin. Immunol., 24(5): 633-39, 2012.
U.S. Pat. No. 4,373,071
U.S. Pat. No. 4,401,796
U.S. Pat. No. 4,458,066
U.S. Pat. No. 4,598,049
U.S. Pat. No. 4,870,287
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,844,905
U.S. Pat. No. 5,846,945
U.S. Pat. No. 5,885,796
U.S. Pat. No. 6,207,156
U.S. Pat. No. 6,225,042
U.S. Pat. No. 6,355,479
U.S. Pat. No. 6,362,001
U.S. Pat. No. 6,410,319
U.S. Pat. No. 6,451,995
U.S. Pat. No. 6,790,662
U.S. Pat. No. 7,070,995
U.S. Pat. No. 7,265,209
U.S. Pat. No. 7,354,762
U.S. Pat. No. 7,446,179
U.S. Pat. No. 7,446,190
U.S. Pat. No. 7,446,191
U.S. Pat. No. 7,666,604
U.S. Pat. No. 8,008,449
U.S. Pat. No. 8,017,114
U.S. Pat. No. 8,119,129
U.S. Pat. No. 8,252,592
U.S. Pat. No. 8,324,353
U.S. Pat. No. 8,329,867
U.S. Pat. No. 8,339,645
U.S. Pat. No. 8,354,509
U.S. Pat. No. 8,398,282
U.S. Pat. No. 8,479,118
U.S. Pat. No. 8,735,553
U.S. Pat. publication No. 2002/131960
U.S. Pat. publication No. 2005/0260186
U.S. Pat. publication No. 2006/0104968
U.S. Pat. publication No. 2009/0004142
U.S. Pat publication No. 2009/0017000
U.S. Pat. publication No. 2011/0008369
U.S. Pat. publication No. 2013/0149337
U.S. Pat. publication No. 2013/287748
U.S. Pat. publication No. 2014/022021
U.S. Pat. publication No. 2014/0294898
Varela-Rohena et al. Nat Med. 14: 1390-1395, 2008.
Wang and Wang, Nat. Biotechnol., 20:149-154, 2002.
Wang et al. J Immunother. 35(9):689-701, 2012.
Wu et al., Cancer, 18(2): 160-75, 2012.
Yee et al. Immunological reviews 257: 250-263. 2014.
Yee et al., J. Immunol. Methods, 261(1-2):1-20, 2002.
Young et al., J. Exp. Med., 183:-11, 1996.
Zwaveling et al., J. Immunol., 169:350-358, 2002.

What is claimed is:

1. An isolated VCX/Y peptide of 35 amino acids in length or less comprising an amino acid sequence having at least 90% sequence identity to SEVEEPLSQ (SEQ ID NO:1)

2. The peptide of claim 1, wherein the VCX/Y peptide is further defined as a VCX3A peptide.

3. The peptide of claim 1 or 2, wherein the peptide comprises an amino acid sequence having at least 95% sequence identity to SEQ ID NO:1.

4. The peptide of any one of claims 1-3, wherein the peptide is 30 amino acids in length or less.

5. The peptide of claim 4, wherein the peptide is 25 amino acids in length or less.

6. The peptide of claim 5, wherein the peptide is 20 amino acids in length or less.

7. The peptide of claim 6, wherein the peptide is 15 amino acids in length or less.

8. The peptide of claim 1, wherein the peptide consists of or consists essentially of SEQ ID NO:1.

9. A pharmaceutical composition comprising the isolated peptide of any one of claims 1- 8 and a pharmaceutical carrier.

10. The composition of claim 9, wherein the pharmaceutical composition is formulated for parenteral administration, intravenous injection, intramuscular injection, inhalation, or subcutaneous injection.

11. The composition of claim 9 or 10, wherein the peptide is comprised in a liposome, lipid-containing nanoparticle, or in a lipid-based carrier.

12. The composition of claim 9, 10, or 11, wherein the pharmaceutical preparation is formulated for injection or inhalation as a nasal spray.

13. An isolated nucleic acid encoding the VCX/Y peptide of any one of claims 1-8.

14. A vector comprising a contiguous sequence comprising or consisting of the nucleic acid of claim 13.

15. A method of promoting an immune response in a subject, comprising administering to the subject an effective amount of the peptide of any one of claims 1-8.

16. The method of claim 15, wherein the subject is diagnosed with cancer.

17. The method of claim 16, wherein the cancer is thymoma, bladder cancer, uterine carcinoma, melanoma, sarcoma, cervix cancer, or head and neck cancer.

18. The method of any one of claims 15-17, wherein the subject is a human.

19. The method of any one of claims 15-18, further comprising administering at least a second anti-cancer therapy.

20. The method of claim 19, wherein the second anti-cancer therapy is selected from the group consisting of a chemotherapy, a radiotherapy, an immunotherapy, hormone therapy, or surgery.

21. The method of claim 20, wherein the immunotherapy comprises one or more immune checkpoint inhibitors.

22. The method of claim 21, wherein the immune checkpoint inhibitor is an anti-PD1 monoclonal antibody.

23. A method of producing VCX/Y-specific immune cells, comprising:
(a) optionally, obtaining a starting population of immune cells; and
(b) contacting a starting population of immune cells with the VCX/Y peptide of any one of claims 1-8, thereby generating VCX/Y-specific immune cells.

24. The method of claim 23, wherein contacting is further defined as co-culturing the starting population of immune cells with antigen presenting cells (APCs), wherein the APCs present the VCX/Y peptide of any one of claims 1-8 on their surface.

25. The method of claim 24. wherein the APCs are dendritic cells.

26. The method of claim 23, wherein the starting population of immune cells are CD8⁺ T cells or CD4⁺ T cells.

27. The method of claim 23, wherein the immune cells are cytotoxic T lymphocytes (CTLs).

28. The method of claim 23, wherein obtaining comprises isolating the starting population of immune cells from peripheral blood mononuclear cells (PBMCs).

29. A VCX/Y-specific immune cell produced according to the method of any one of claims 23-28.

30. A pharmaceutical composition comprising the VCX/-specific immune cells produced according to the method of any one of claims 23-28.

31. A method of treating cancer in a subject, comprising administering an effective amount of VCX/Y-specific immune cells of claim 29.

32. The method of claim 31, wherein the immune cell is a T cell, peripheral blood lymphocyte, NK cell, invariant NK cell, NKT cell, mesenchymal stem cell (MSC), induced pluripotent stem (iPS) cell, or mixture thereof.

33. The method of claim 31 or 32, wherein the immune cell is isolated from the umbilical cord.

34. The method of any one of claims 31-33, wherein the immune cell is autologous or allogeneic with respect to the subject.

35. The method of claim 31, wherein the immune cell is a CD8⁺T cell, CD4⁺T cell, or γδT cell.

36. The method of any one of claims 31-35, wherein the cancer is thymoma, bladder cancer, uterine carcinoma, melanoma, sarcoma, cervix cancer, or head and neck cancer.

37. The method of any one of claims 31-36, wherein the subject is a human.

38. The method of any one of claims 31-37, further comprising lymphodepletion of the subject prior to administration of the VCX/Y -specific immune cells.

39. The method of claim 38, wherein lymphodepletion comprises administration of an effective amount of cyclophosphamide and/or fludarabine,

40. The method of any one of claims 31-39, further comprising administering at least a second therapeutic agent to the subject.

41. The method of claim 40, wherein the at least a second therapeutic agent comprises chemotherapy, immunotherapy, surgery, radiotherapy, hormone therapy, and/or biotherapy.

42. The method of claim 41, wherein the VCX/Y-specific immure cells and/or at least a second therapeutic agent are administered intravenously, intraperitoneally, intratracheally, intratumorally, intramuscularly, endoscopically, intralesionally, percutaneously, subcutaneously, regionally, or by direct injection or perfusion.

43. The method of any one of claims 31-42, wherein the subject is determined to have cancer cells that express a protein of the VCX/Y family.

44. The method of claim 43, wherein the protein is VCX3A.
